(19) United States
(12) Patent Application Publication
(10) Pub. No.: US 2020/0258618 A1
(43) Pub. Date: Aug. 13, 2020

(54) PATIENT FLOW

(71) Applicant: KONINKLIJKE PHILIPS N.V., Eindhoven (NL)

(72) Inventors: SOPHIA HUAI ZHOU, CAMBRIDGE, MA (US); LARRY JAMES ESHELMAN, OSSINING, NY (US); MINNAN XU, CAMBRIDGE, MA (US); JOSEPH JAMES FRASSICA, GLOUCESTER, MA (US); JOHN CUSTER RYAN, WABAN, MA (US); ANDREW FRANKLIN ARTHUR, HOLLIS, NH (US)

(21) Appl. No.: 16/776,605

(22) Filed: Jan. 30, 2020

Related U.S. Application Data
(60) Provisional application No. 62/802,733, filed on Feb. 8, 2019.

(51) Int. Cl. G16H 40/20; G16H 10/60; G16H 40/63; G16H 50/30; G16H 70/00; G16H 70/20; G16H 50/50; G06N 20/00; A61B 5/00; A61B 5/0205; A61B 5/1455

(52) U.S. Cl. CPC G16H 40/20; G16H 10/60; G16H 40/63; G16H 50/30; G16H 70/00; G16H 70/20; A61B 5/024; G06N 20/00; A61B 5/7264; A61B 5/02055; A61B 5/14551; A61B 5/7267; A61B 5/725; G16H 50/50

(57) ABSTRACT

Methods and systems for monitoring patient physiological status. The system may include a source of vital sign measurements for a patient, a trained machine learning model that receives the vital sign measurements and provides an output related to the physiological status of the patient, and an interface configured to present the output to an operator. The method may include receiving, at a trained machine learning model, at least one physiological measurement, demographic information point, or treatment plan for a patient, providing, using the trained machine learning model, an output relating to the physiological status of the patient, and presenting, using an interface, the output to an operator.

### CROSS-REFERENCE TO PRIOR APPLICATIONS

This application claims the benefit of or priority of U.S. Provisional patent application Ser. No. 62/802,733, filed Feb. 8, 2019, all of which are incorporated herein in whole by reference.

### TECHNICAL FIELD

Embodiments described herein generally relate to systems and methods for monitoring patient physiological status and, more particularly but not exclusively, to systems and methods for improved patient flow.

### BACKGROUND

Hospitals are in urgent need for solutions to improve patient care quality, patient safety, and reduce cost. Hospitals worldwide experience problems with high occupancy rates, overcrowding, communication burdens, slow hospital discharge, risk of patient readmission, coordination in medical device utilization, resource planning, and costs. The yield from the investment in healthcare in the United States is low when compared to other countries. The United States spends almost twice as much per capita on healthcare as other industrialized nations, yet in many cases has inferior health outcomes to show for it. While the causes of this imbalance are varied, it is clear that health systems in the United States must find ways to provide better care to broader populations while not increasing total costs further.

Hospitals are designed for an average 85% occupancy, but current hospital occupancy rates can be 100-120% on average in the United States. Occupancies greater than 85% introduce problems with shortages of beds, nursing staff, medical equipment, and scheduling issues. Overcrowding in the emergency department, radiology department, intensive care unit (ICU), and operating rooms are constant struggles for many hospitals.

In some hospitals, patients must spend upwards of six hours at the emergency room waiting for medical care. Many visits are not for urgent conditions and some may leave the emergency department without being seen by a medical professional. Hospitals not only lose revenue opportunities but also may also be penalized if patients leave without being seen by a medical professional.

Moreover, when patients are in the hospital, it may be difficult to escalate patient care when a patient deteriorates or becomes unstable, especially if the patient must be transferred between wards of a hospital. At the same time, the current practice of discharging a patient can be a complex process, wherein multiple medical professionals must weigh the risks of keeping a patient too long in a hospital against the risks of sending a patient away too quickly. If a patient returns to the hospital within a 30-day window after discharge, the hospital may be penalized and may lose revenue. However, a patient kept in a hospital too long runs an increased risk of hospital-acquired infection and wasting valuable hospital resources.

At the population level, hospitals attempt to minimize unnecessary testing and optimize each hospital patient's transitions from one department to another to reduce waste and reduce the risk of associated hospital acquired infections. At the patient level, decisions for care escalation and patient discharge are critical for patient post-hospital recovery. If these are not properly managed, the risk of patient readmission increases substantially.

A need exists, therefore, for methods and systems that overcome the above disadvantages of monitoring and predicting the physiological status of a patient.

### SUMMARY

This summary is provided to introduce a selection of concepts in a simplified form that are further described below in the Detailed Description section. This summary is not intended to identify or exclude key features or essential features of the claimed subject matter, nor is it intended to be used as an aid in determining the scope of the claimed subject matter.

In one aspect, embodiments relate to a system for monitoring patient physiological status. The system includes a source of vital sign measurements for a patient; a trained machine learning model that receives the vital sign measurements and provides an output related to the physiological status of the patient; and an interface configured to present the output to an operator.

In some embodiments, the vital sign measurements are selected from the group consisting of heart rate, systolic blood pressure, body temperature, peripheral capillary oxygen desaturation, and respiratory rate. In some embodiments, the system further includes a source of facility information for training machine learning models. In some embodiments, the system further includes a filter for smoothing the output of the trained machine learning model. In some embodiments, the filter is a median filter. In some embodiments, the system further includes a lead/lag indicator that takes the output values exceeding the smoothed output for a given window size, weights them, and provides the maximum of the weighted scores. In some embodiments, the output comprises a transition score. In some embodiments, the output further comprises a confidence interval score.

In another aspect, embodiments relate to a method for monitoring patient physiological status. The method includes receiving, at a trained machine learning model, at least one physiological measurement, demographic information point, or treatment plan for a patient; providing, using the trained machine learning model, an output relating to the physiological status of the patient; and presenting, using an interface, the output to an operator.

In some embodiments, the method includes receiving, at the trained learning machine model, at least one physiological measurement selected from the group consisting of heart rate, systolic blood pressure, body temperature, peripheral capillary oxygen desaturation, and respiratory rate. In some embodiments, the method further includes retraining the machine learning model using a source of facility information. In some embodiments, the method further includes smoothing the output of the trained machine learning model using a filter. In some embodiments, the method further includes taking the output values exceeding the smoothed output for a given window size, weighting them, and providing the maximum of the weighted scores. In some embodiments, the method further includes receiving an expected length of stay for the patient, wherein the length of stay terminates at a discharge time, and presenting the output within 48 hours prior to the discharge time. In some embodiments, the method further includes evaluating the patient for discharge within 48 hours of the expected length of stay of the patient and creating a conditional discharge order for the patient.

In some embodiments, the physiological status of the patient comprises the predicted stability of the patient over a subsequent time period. In some embodiments, the method further includes receiving an expected length of stay for the patient, wherein the length of stay terminates at a discharge time and wherein the patient is an observation patient; determining if a discharge order has been ordered for the patient; and if the discharge order has not been ordered, evaluating the output to determine if the patient should be evaluated for discharge.

In some embodiments, the method further includes evaluating the output; determining, based on the output, that the patient should be evaluated for a discharge order; and evaluating the patient for a discharge order.

In yet another aspect, embodiments relate to a non-transitory computer-readable medium comprising computer-executable instructions for performing a method for monitoring patient physiological status. The medium includes computer-executable instructions for receiving, at a trained machine learning model, vital sign measurements for a patient; computer-executable instructions for providing, using the trained machine learning model, an output relating to the physiological status of the patient; and computer-executable instructions for presenting, using an interface, the output to an operator.

In some embodiments, the medium further includes computer-executable instructions for retraining the machine learning model using a source of facility information.

### BRIEF DESCRIPTION OF DRAWINGS

Non-limiting and non-exhaustive embodiments of the invention are described with reference to the following figures, wherein like reference numerals refer to like parts throughout the various views unless otherwise specified.

FIG. 1 illustrates a method to calculate transition scores in accordance with one embodiment;

FIG. 2 illustrates a graph representing the raw and smoothed transition scores of a patient calculated over time in accordance with one embodiment;

FIG. 3 illustrates a graph representing the raw, short-term smoothed, and long-term smoothed transition scores of a patient calculated over time in accordance with one embodiment;

FIGS. 4A and 4B illustrate graphs representing sets of patient transition scores with average positive and negative slopes, respectively, in accordance with one embodiment; and

FIG. 5 illustrates the transfer of patients by transition scores through hospital departments and discharge in accordance with one embodiment.

### DETAILED DESCRIPTION

Various embodiments are described more fully below with reference to the accompanying drawings, which form a part hereof, and which show specific exemplary embodiments. However, the concepts of the present disclosure may be implemented in many different forms and should not be construed as limited to the embodiments set forth herein; rather, these embodiments are provided as part of a thorough and complete disclosure, to fully convey the scope of the concepts, techniques and implementations of the present disclosure to those skilled in the art. Embodiments may be practiced as methods, systems or devices. Accordingly, embodiments may take the form of a hardware implementation, an entirely software implementation or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one example implementation or technique in accordance with the present disclosure. The appearances of the phrase “in one embodiment” in various places in the specification are not necessarily all referring to the same embodiment. The appearances of the phrase “in some embodiments” in various places in the specification are not necessarily all referring to the same embodiments.

Some portions of the description that follow are presented in terms of symbolic representations of operations on non-transient signals stored within a computer memory. These descriptions and representations are used by those skilled in the data processing arts to most effectively convey the substance of their work to others skilled in the art. Such operations typically require physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices. Portions of the present disclosure include processes and instructions that may be embodied in software, firmware or hardware, and when embodied in software, may be downloaded to reside on and be operated from different platforms used by a variety of operating systems.

The present disclosure also relates to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each may be coupled to a computer system bus. Furthermore, the computers referred to in the specification may include a single processor or may be architectures employing multiple processor designs for increased computing capability.

The processes and displays presented herein are not inherently related to any particular computer or other apparatus. Various general-purpose systems may also be used with programs in accordance with the teachings herein, or it may prove convenient to construct more specialized apparatus to perform one or more method steps. The structure for a variety of these systems is discussed in the description below. In addition, any particular programming language that is sufficient for achieving the techniques and implementations of the present disclosure may be used. A variety of programming languages may be used to implement the present disclosure as discussed herein.

In addition, the language used in the specification has been principally selected for readability and instructional purposes and may not have been selected to delineate or circumscribe the disclosed subject matter. Accordingly, the present disclosure is intended to be illustrative, and not limiting, of the scope of the concepts discussed herein.

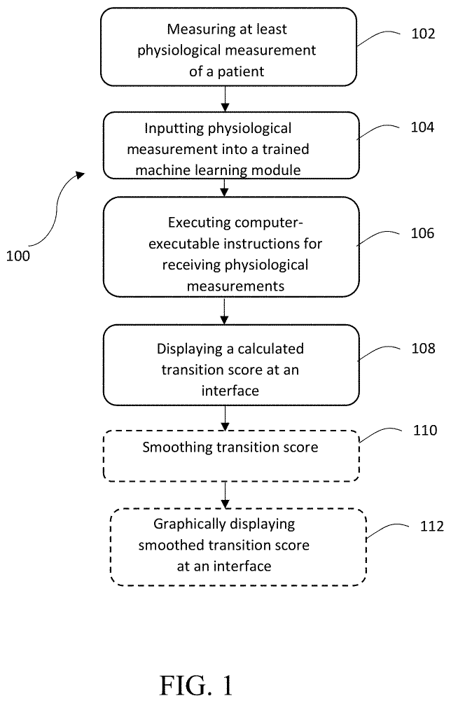

As mentioned previously, embodiments relate to systems and methods for monitoring the physiological status of a patient. FIG. 1 illustrates a method 100 for calculating the transition score of a patient. In some embodiments, the method may include measuring at least one physiological measurement of a patient 102. Physiological measurements may include the vital sign measurements of a patient, such as at least one of a heart rate, systolic blood pressure, body temperature, peripheral capillary oxygen desaturation, and respiratory rate of a patient. In some embodiments, a system employing the method depicted in FIG. 1 may use a plurality of sources, including a heart rate monitor, a breathing rate monitor, respirators, thermometers, and oxygen detectors to measure the vital signs of a patient.

In some embodiments, the measured physiological measurements may be inputted into a trained machine learning module 104. In some embodiments, demographic information and treatment plans for a patient may also be inputted into the trained machine learning module. For example, in some embodiments, the system may receive input about at least one of the age, weight, size, and medication for a patient. In some embodiments, the system may use the input(s) to determine a transition score of the patient.

In some embodiments, the system may execute computer-executable instructions for receiving physiological measurements 106. For example, the system may have an algorithm for calculating an output related to the physiological status of the patient based on the received input. This output may be referred to as a transition score. The transition score may be based on at least one patient vital sign measurement, patient demographic information, and a patient treatment plan. The trained machine learning module may also use a source of facility information for training and machine learning. The module may include computer-executable instructions for retraining the machine learning model using a source of facility information in some embodiments.

The algorithm for the transition score may use inputs of the vital sign measurements and coefficients to determine the transition score of a patient. In some embodiments, the coefficients may be derived through machine learning. The hospital may also be able to adjust the calculations, including the coefficients used in the algorithm, periodically or continuously by inputting more information available from the hospital. The hospital may also be able to adjust the results of a transition score to comply with hospital protocol.

In some embodiments, the transition score may be continuously calculated and updated. In some embodiments, the transition score of a patient may be periodically updated. The transition score refresh rate may be determined by the input data frequency 104 for the patient. In some embodiments, the transition score include probability calculations and timeline calculations, showing the likelihood that a particular score accurately forecasts the physiological status of a patient over time. In some embodiments, the system may use machine learning to adjust the probability calculations and the timeline calculations after receiving input about the accuracy of previous calculations 106.

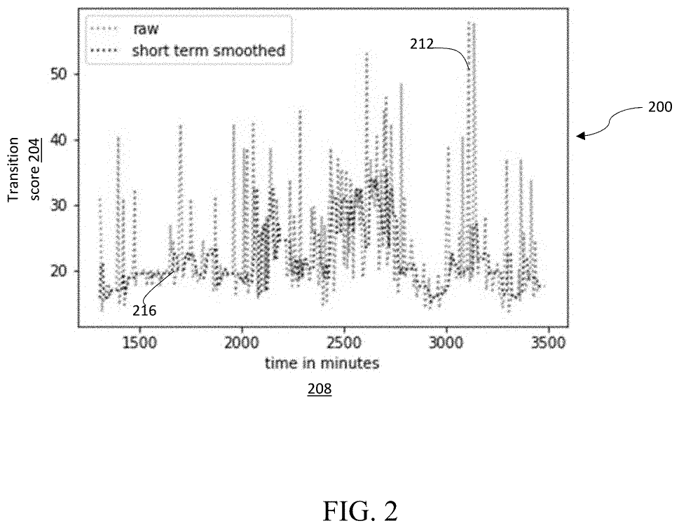

In some embodiments, the transition score of a patient may be reported and displayed on an interface of a device in a medical care unit 108. In some embodiments, devices may be linked with an information system to access the transition score of a patient and display the patient scores on an interface. In some embodiments, the transition score may be displayed with at least one vital sign of a patient 108. The transition score may be presented to an operator in graph form at an interface to graph the progress of a patient over time. In some optional embodiments, the method may include smoothing the transition score 110 and graphically displaying the smoothed transition score 112 at an interface. Some embodiments may use a median filter to smooth transition score values over a period of time to obtain a more stable transition score value for reporting and displaying to an operator. As seen in FIG. 2, in some embodiments, the smoothed transition score 112 may be displayed simultaneously with a raw calculated transition score 108.

In some embodiments, the transition score may be used to represent a patient's physiological status and, through charting and outputting the transition score 108, an operator or computer may determine the status change of a patient over time. For example, a decreased transition score may indicate the progression of a patient physiological condition and an increased transition score may indicate the regression of a patient physiological condition. A regression of a patient physiological condition may indicate that the patient care needs to be escalated, whereas a progression of a patient physiological condition may indicate that the patient may be eligible for discharge.

Calculating a transition score may allow clinicians to monitor patient change over time and across an entire hospital stay while simultaneously allowing clinicians to standardize the comparison of patient physiological status. Each patient may have a calculated and reported transition score during a hospital stay. In some embodiments, the system may use transition scores to give a care team insight into where a patient should be transitioned to, when they will be ready to be transitioned, and the probability of either or both of these outcomes.

For hospital managers, in some embodiments, the transition score may be used to predict future aggregate demand. Hospital managers may be able to determine the aggregate demand for a resource in 6, 12, and 24 hours so that the manager may better aggregate capacity to meet the future potential demand. For example, hospital managers may be able to determine that two ventilators will not be needed in the ICU in 12 hours for two patients and will be needed for a future patient being transitioned from the emergency room. In some embodiments, the system may be able to predict the cumulative demand for devices more accurately than a single patient's need for a device within the same time span. For instance, if the hospital has a history of a high prevalence of patients with respiratory complications in pulmonary ICUs, more ventilators might be needed during the peak season. Similarly, the hospital may need more telemetry ECG monitors if higher cardiac complications are predicted when a community is facing a natural disaster such as wildfire or a hurricane.

Furthermore, the transition scores may indicate and forecast the percentage or number of patients that may be discharged in a certain time period. The transition scores may indicate and forecast the number or percentage of patients that may need to be escalated to high-level care. The transition scores may be associated with a confidence level in a prediction or forecast on an individual patient basis, department basis, or hospital basis in some embodiments.

In some embodiments, the transition score calculations may improve cost and revenue for the hospital because shorter and more efficient length of stay may allow a hospital to see more patients in a given amount of time, reduce the risk of hospital acquired infections, and may improve patient satisfaction. Transition score predictions may help assess patients upon hospital entry, discharge a patient faster with a higher degree of confidence with respect to patient safety, and may reduce overcrowding and bottlenecking in hospital departments. Furthermore, standardizing transition scores based on vital signs may help with objective evaluation of a patient and reduce readmission risk by more accurately predicting future physiological stability of a patient. Improving transition score algorithms with machine learning may also help with the accuracy and effectiveness of medical decision making with respect to patient transition.

For example, FIG. 2 illustrates a graph 200 representing the transition score 204 of a patient calculated over time 208, with one set of dots showing the raw transition score 212 of a patient at a time and a second set of dots showing the filtered scores 216 based on a smoothing method, described in further detail below. In some embodiments, the raw score 212 may be based on a combination of vital signs, including but not limited to a patient's heart rate, systolic blood pressure, body temperature, peripheral oxygen capillary desaturation, and respiratory rate. In some embodiments, the system may calculate the raw score 212 using at least one demographic information point, or treatment plan for a patient.

Because of the nature of human physiology, the values of vital sign measurements may have minor variations over time. Summed multiple variations from all vital signs may also have minor variations. The minor variations may not be meaningful when determining the physiological status of a patient over time.

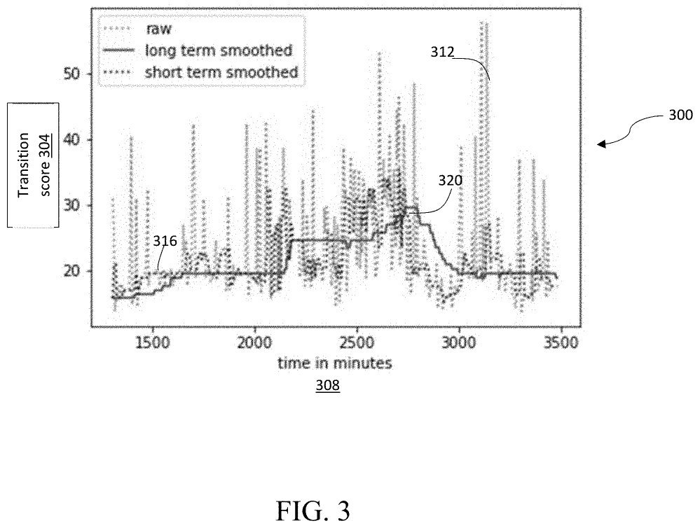

In some embodiments, the system may apply a median filter to calculated transition scores and may display the filtered result in a graph such as the graph 300 depicted in FIG. 3. In some embodiments, the transition score 304 over time 308 may be shown as a raw score 312, a short-term smoothed score 316, or a long-term smoothed score 320. In some embodiments, a short-term smoothed score 316 may average the variations in transition scores over a period of five minutes. In some embodiments, the system may only smooth transition scores 316, 320 if three or more transition score values are available in a given time window.

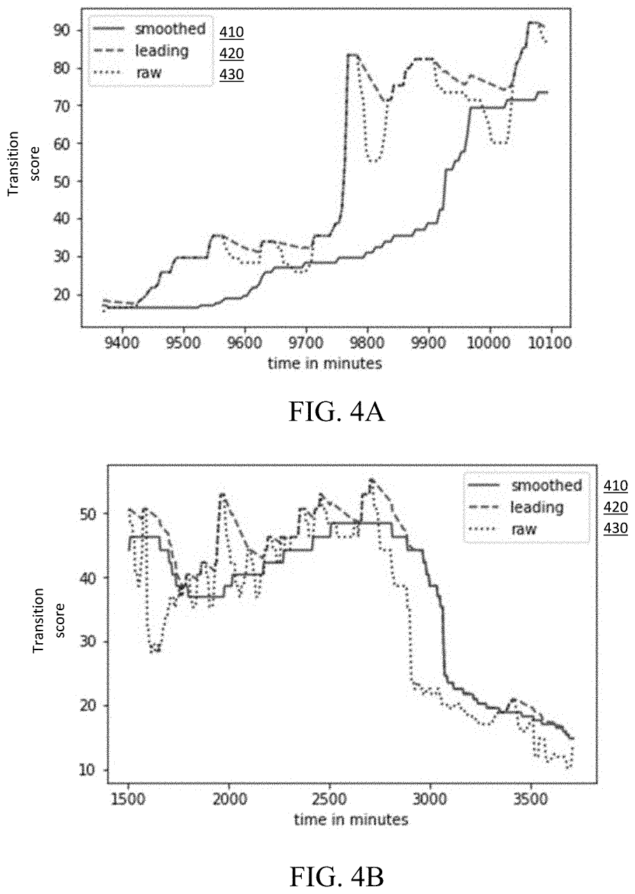

The slope between transition score graph points may be negative or positive. For example, FIG. 4A shows a set of transition scores with an average positive slope and FIG. 4B shows a set of transition scores with an average negative slope. In some embodiments, a negative slope indicates a decreased transition score and a positive slope indicates an increased transition score. An increased transition score may indicate the regression of a patient and a decreased transition score may indicate the improvement of a patient. The patient corresponding to FIG. 4A may be transitioned to a more intensive care unit, whereas the patient corresponding to FIG. 4B may be transitioned to a less intensive care unit or may be trending towards a discharge order.

In some embodiments, the system may report a confidence interval score or confidence range associated with a transition score. The confidence range may be estimated based on data availability and data frequency of a patient. In some embodiments, the confidence range may be based on a long-term indicator and a lead/lag indicator. A lead/lag indicator may be a combination of a lead indicator of deterioration and a lag indicator of improvement. For example, in some embodiments, the potential future deterioration of a patient may be prioritized over the potential future improvement of a patient, such that the lead indicator of deterioration may be prioritized in time over the lag indicator of improvement. In a hospital scenario, the algorithm calculating a confidence range associated with a transition score may be more vigilant about patient deterioration than improvement. In some embodiments, the lead/lag indicator may be equal to or greater than the long-term indicator at any point in time. In some embodiments, the lead/lag indicator may take the raw transition score output values exceeding the smoothed output or long-term indicator for a given window size, weigh the outputs, and provide the maximum of the weighted scores.

In some embodiments, a long-term indicator may be a median filter, a moving average or other type of smoothing filter. The lead/lag indicator may incorporate scores above the smoothed score for a given time period, weigh the scores with more weight given to the more recent scores, and then may calculate the maximum weighted score. In some embodiments, if the raw scores are above the long-term indicator and are rising, the lead/lag indicator may follow the raw scores. The raw scores will lead when the scores indicate the deterioration of a patient in some embodiments. In some embodiments, if the raw scores are below the long-term indicator and are falling, indicating the improvement of a patient, the lead/lag indicator may fall more slowly than the raw scores. The long-term indicator, rather than the most recent positive scores, may lead when the scores indicate the improvement of a patient in some embodiments.

In FIG. 4A, the transition score of the patient trends upward toward a possible step-up transfer. The raw scores 430 may be used to calculate the long-term indicator 410 and the lead/lag indicator 420. In FIG. 4A, the lead/lag indicator 420 differs from the long-term indicator 410 at approximately minute 9430, wherein the lead/lag indicator 420 follows the rising raw scores 430. At approximately minute 9750, the lead/lag indicator 420 rises again, following the rising raw scores 430. The lead/lag indicator 420 lags when the raw score 430 improves at approximately minute 9800, remaining more stable over time when the raw score 430 increases at minute 9850. By around minute 9950, the long-term indicator 410 increases and the gap between the lead/lag indicator 420, the raw score 430, and the long-term indicator 410 decreases

In some embodiments, a large gap between the long-term indicator 410 and the lead/lag indicator 420 may indicate a high degree of uncertainty. Furthermore, in some embodiments, if the long-term indicator 410 is very high, a step-up transfer for a patient should be considered. In some embodiments, a low lead/lag indicator 420 may indicate that a step-down transfer should be considered.

In some embodiments, the confidence range is the range between the long-term indicator value 410 and the lead/lag indicator value 420 at any point in time. For example, in FIG. 4A, at minute 9800, the confidence range is 31 to 79 (and the raw value is 58). For the last point in the plot, minute 10100, the confidence range is 74 to 90 (and the raw value is 87).

In some embodiments, the transition score may be localized by using an adaptive transition score capability for a hospital. For example, some embodiments may use devices supported by the Clinical and Operational Command Center (CLOC) information system to display a transition score. In some embodiments, the CLOC operators may apply tools embedded in the CLOC solution to generate a new transition score based on new information from a local hospital. The system may then feed the new information into the required tool and a new transition score may be generated by the system. In some embodiments, an operator may choose to switch from a localized transition score to a transition score using factory configurations and back at any time.

In some embodiments, the transition score, in conjunction with the confidence range, may be used to predict patient physiological status change to better or worse hours or even days before a critical event occurs. Clinicians may be prepared to prevent such events, such as hemodynamic complication or a respiratory complication. The clinicians and families can also be prepared for early discharge if fast post-surgical recovery is anticipated.

For example, a patient admitted in the emergency department with a high transition score may be moved to the ICU or other department within hours of admission. After treatment, the transition score of the patient may decrease such that the patient may be ready for discharge sooner than predicted when the patient entered the emergency department. In some embodiments, a patient may be admitted directly to an ICU or general ward as a hospital referral and the transition score of the patient may be tracked from the initial admission at a separate facility, through transportation to the hospital and admission to the hospital.

In some embodiments, the transition score may indicate what ward a patient may be transferred to. For example, a transition score of above 20 may indicate that the patient should be at least in the ICU. A transition score of below 20 may indicate that the patient should be in a general ward. A transition score of below 5 may indicate that the patient should be discharged from the hospital within a certain period of time.

In some embodiments, the transition score may be adjusted based on at least one of the patient setting and a discharge-from-department checklist. For example, a patient may have a transition score of between 10 and 30 when they enter the emergency department. When a patient's transition score decreases below 10, the patient may be transitioned to the ICU. At the ICU, the patient's transition score may be re-calculated based on the new patient setting such that the patient's transition score may be between 10 and 30 in the ICU. If the patient's transition score decreases below 10 in the ICU, the patient may be transferred to a general ward, where the transition score would be recalibrated again in accordance with some embodiments.

In some embodiments, the transition score may be updated after medical professionals complete a discharge-from-department checklist. In some embodiments, a discharge-from-department checklist may be a set of steps needed to be completed before the patient can be transferred, independent of the clinical status of the patient. If the adjusted transition score is lower than the transition score, clinicians may focus on completing the patient's discharge-from-department checklist.

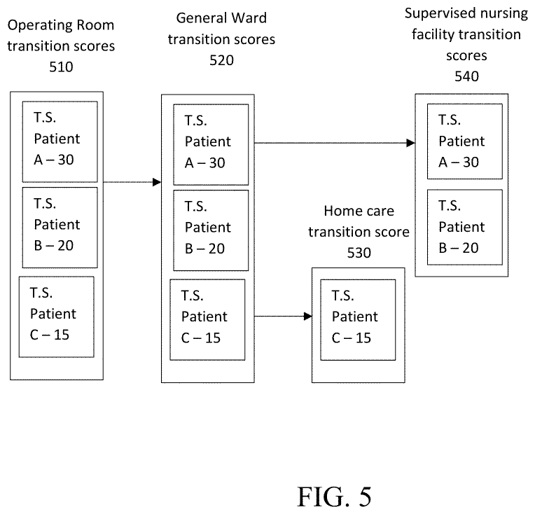

In some embodiments, the distribution of transition scores in a hospital department may assist in patient flow planning. For example, in FIG. 5, three patients may have initial transition scores between 15 and 30 in the operating room 510. The transition scores may be adjusted based on patient settings when the patients are transitioned from the operating room to the general ward in some embodiments. For patient C, the transition score may be low enough that patient C may transfer directly from the general ward 520 to home care 530 in some embodiments. For patients A and B, the transition scores in the general ward may be too high to directly discharge the patients to home care. In some embodiments, patients with transition scores above 15 may be transferred from the general ward to a supervised nursing facility 540.

Some embodiments may use transition scores to determine potential discharge orders for a patient. For example, in some embodiments, a patient may enter the hospital with an expected length of stay terminating at a predicted discharge time. In some embodiments, the patient may be an observation patient. In some embodiments, the system may use transition scores of a patient within 48 hours of a predicted discharge time to determine the accuracy of the initially predicted discharge time. In some embodiments, the system may evaluate the patient for discharge within 48 hours of the expected length of stay and may create a conditional discharge order for the patient. A conditional discharge order may speed the discharge process once the patient qualifies for discharge.

In some embodiments, the system may calculate the predicted stability of the patient over a subsequent time period when determining the physiological status of the patient. For example, the system may use the transition scores to predict patient stability 24 hours, 48 hours, or 30 days from the time of discharge. As another example, the system may use transition scores to predict patient stability and determine the probability of the patient's need for medical equipment within a subsequent time period.

In some embodiments, the system may periodically determine if a discharge order has been ordered for a patient. If the discharge order has not been ordered, the system may evaluate the output related to the vital signs of the patient to determine if the patient should be evaluated for discharge. In some embodiments, the system may evaluate the transition scores of the patient in a recent time window to determine if the patient should be evaluated for discharge. In some embodiments, the system may output the transition scores to the interface of a device and an operator may determine if the patient should be evaluated for discharge. If the patient should be evaluated for discharge, an operator or the system may subsequently evaluate the patient. In some embodiments, the evaluation may be coupled to a confidence level score.

In some embodiments, knowledge of the number of patients who may need to be transferred from the general ward to supervised nursing facilities may help with the management of patient flow monitoring. For example, at the population level in a department, a service, or in the entire hospital, this information may help administrators in each department, service or hospital better manage patient flow, bed occupancy, equipment utilization, and radiology and operating room scheduling optimization.

At a department level, the distribution of the patient populations' transition scores may help to assess the global hospital capacity. For example, a natural distribution of transition scores may have a large population of patients with a low transition score, a smaller population of patients with a medium transition score, and an even smaller population of patients with a high transition score. Patients with a high transition score may need more attention, equipment, and clinician time than patients with low transition scores. If the distribution of transition scores is even, such that an equal number of patients have high, medium, and low transition scores, the patient flow may create burdens of bed occupancy in downstream departments, staff shortages, and equipment shortages. Moreover, the burdens may be exacerbated if there are more patients with higher transition scores than lower transition scores. In some embodiments, the transition scores may be fed into algorithms to predict supply and demand for departments in a hospital. In some embodiments, this transition score forecasting at a department level may be coupled to forecast confidence levels.

Some embodiments may use cumulative cost-weighted and activity adjusted transition scores. In some embodiments, hourly hospital costs may be estimated for each department based on patient waiting time. Waiting times may cause sequential waste. For example, if a patient is waiting in a bed for a C.T. scan before discharge, the patient may unnecessarily occupy a bed another patient may need. This may cause delays to transition patients from other departments and may result in patients in the emergency department leaving without being seen. In some embodiments, one delay may add to lost revenue opportunities in many departments for the hospital.

Some embodiments may use transition scores to operate a central flow manager. In some embodiments, the central flow manager may help a hospital to re-prioritize equipment, predict future needs, and reconfigure to reduce wait times in the hospital. In some embodiments, the central flow manager may output calculations and future need predictions alongside calculated confidence scores to an interface. In some embodiments, the transition score may be shared by all acute care information systems, including but not limited to ICCA (IntelliVue Critical Care and Anesthesia), IGS (IntelliVue Guardian System), eCM (eCare manager), TASY EMR system, and PIICix (Intelli Space Information Center).

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Embodiments of the present disclosure, for example, are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrent or the blocks may sometimes be executed in the reverse order, depending upon the functionality/acts involved. Additionally, or alternatively, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Having described several example configurations, various modifications, alternative constructions, and equivalents may be used without departing from the spirit of the disclosure. For example, the above elements may be components of a larger system, wherein other rules may take precedence over or otherwise modify the application of various implementations or techniques of the present disclosure. Also, a number of steps may be undertaken before, during, or after the above elements are considered.

Having been provided with the description and illustration of the present application, one skilled in the art may envision variations, modifications, and alternate embodiments falling within the general inventive concept discussed in this application that do not depart from the scope of the following claims.

What is claimed is:

1. A system for monitoring patient physiological status, the system comprising:
a source of vital sign measurements for a patient;
a trained machine learning model that receives the vital sign measurements and provides an output related to the physiological status of the patient; and
an interface configured to present the output to an operator.

2. The system of claim 1 wherein the vital sign measurements are selected from the group consisting of heart rate, systolic blood pressure, body temperature, peripheral capillary oxygen desaturation, and respiratory rate.

3. The system of claim 1 further comprising a source of facility information for training machine learning models.

4. The system of claim 1 further comprising a filter for smoothing the output of the trained machine learning model.

5. The system of claim 4 wherein the filter is a median filter.

6. The system of claim 4 further comprising a lead/lag indicator that takes the output values exceeding the smoothed output for a given window size, weights them, and provides the maximum of the weighted scores. The system of claim 1, wherein the output comprises a transition score.

8. The system of claim 7, wherein the output further comprises a confidence interval score.

9. A method for monitoring patient physiological status, the method comprising:
receiving, at a trained machine learning model, at least one physiological measurement, demographic information point, or treatment plan for a patient;
providing, using the trained machine learning model, an output relating to the physiological status of the patient; and
presenting, using an interface, the output to an operator.

10. The method of claim 9 comprising receiving, at the trained learning machine model, at least one physiological measurement selected from the group consisting of heart rate, systolic blood pressure, body temperature, peripheral capillary oxygen desaturation, and respiratory rate.

11. The method of claim 9 further comprising retraining the machine learning model using a source of facility information.

12. The method of claim 9 further comprising smoothing the output of the trained machine learning model using a filter.

13. The method of claim 12 further comprising taking the output values exceeding the smoothed output for a given window size, weighting them, and providing the maximum of the weighted scores.

14. The method of claim 9, further comprising receiving an expected length of stay for the patient, wherein the length of stay terminates at a discharge time, and presenting the output within 48 hours prior to the discharge time.

15. The method of claim 9, further comprising evaluating the patient for discharge within 48 hours of the expected length of stay of the patient and creating a conditional discharge order for the patient.

16. The method of claim 9, wherein the physiological status of the patient comprises the predicted stability of the patient over a subsequent time period.

17. The method of claim 16, further comprising:
receiving an expected length of stay for the patient, wherein the length of stay terminates at a discharge time and wherein the patient is an observation patient;
determining if a discharge order has been ordered for the patient; and
if the discharge order has not been ordered, evaluating the output to determine if the patient should be evaluated for discharge.

18. The method of claim 9, further comprising:
evaluating the output;
determining, based on the output, that the patient should be evaluated for a discharge order; and
evaluating the patient for a discharge order.

19. A non-transitory computer-readable medium comprising computer-executable instructions for performing a method for monitoring patient physiological status, the medium comprising:
computer-executable instructions for receiving, at a trained machine learning model, vital sign measurements for a patient;
computer-executable instructions for providing, using the trained machine learning model, an output relating to the physiological status of the patient; and
computer-executable instructions for presenting, using an interface, the output to an operator.

20. The medium of claim 19 further comprising computer-executable instructions for retraining the machine learning model using a source of facility information.
